(19) United States
(12) Patent Application Publication
(10) Pub. No.: US 2021/0358569 A1
(43) Pub. Date: Nov. 18, 2021

(54) METHODS AND SYSTEMS FOR ASSESSING MICROSATELLITE INSTABILITY

(71) Applicant: Lexent Bio, Inc., Cambridge, MA (US)

(72) Inventors: Alexander De Jong ROBERTSON, San Francisco, CA (US); Nicole Jacinda LAMBERT, San Francisco, CA (US); Haluk TEZCAN, San Francisco, CA (US); Ram YALAMANCHILI, San Francisco, CA (US); Neil PETERMAN, San Francisco, CA (US); Rohith Kannappan SRIVAS, San Francisco, CA (US)

(21) Appl. No.: 17/275,160

(22) PCT Filed: Sep. 13, 2019
(86) PCT No.: PCT/US19/51138

§ 371 Date: Mar. 10, 2021

Related U.S. Application Data
(60) Provisional application No. 62/731,718, filed on Sep. 14, 2018.

(51) Int. Cl. G16B 40/00; G16H 20/40

(52) U.S. Cl. CPC G16B 40/00; G16H 20/40

(57) ABSTRACT

The invention disclosed herein generally relates to methods of assessing microsatellite instability in a subject. In an aspect, the present disclosure provides a computer-implemented method of assessing microsatellite instability of a subject, comprising: obtaining a quantitative measure of a plurality of microsatellite repeat elements from a blood sample of a subject; processing the plurality of quantitative measures to obtain a statistical measure of deviation of the plurality of quantitative measures; and detecting a presence of the microsatellite instability (MSI) of the subject when the statistical measure of deviation of the plurality of quantitative measures satisfies a predetermined criterion, or detecting an absence of the microsatellite instability (MSI) of the subject when the statistical measure of deviation of the plurality of quantitative measures does not satisfy the predetermined criterion.

### CROSS-REFERENCE

This application claims the benefit of U.S. Provisional Patent Application No. 62/731,718, filed Sep. 14, 2018, which is entirely incorporated herein by reference.

### BACKGROUND

Microsatellite instability (MSI) may generally refer to a condition of genetic predisposition to mutation which may result from impaired DNA mismatch repair (MMR) in a subject. In subjects with MSI, cells with abnormally functioning MMR may accumulate errors during DNA replication, resulting in mutated microsatellite fragments, or repeated DNA sequences. MSI may play a significant role in many types of cancers, such as colon cancer, gastric cancer, endometrial cancer, ovarian cancer, hepatobiliary tract cancer, urinary tract cancer, brain cancer, and skin cancers. For example, MSI is a good marker for detection of hereditary nonpolyposis colorectal cancer (HNPCC) or Lynch syndrome, an autosomal dominant genetic condition that has a high risk of colon cancer and other types of cancers. In addition, microsatellite status may be indicative of a prognosis of a subject for cancer treatments. For example, MSI studies in colon cancer patients have indicated better prognosis for MSI-high patients (MSI-H) as compared to patients with MSI-low (MSI-L) or microsatellite stable (MSS) tumors.

### SUMMARY

Methods, systems, and media are provided herein for assessing microsatellite instability (MSI) of a subject, such as a patient with cancer, by analyzing a blood sample of the subject. Microsatellite instability (MSI) may be assessed and/or monitored by analyzing tumor DNA (e.g., from cell-free DNA) from a sample of a subject in a plurality of genetic loci corresponding to microsatellites comprising mononucleotides and dinucleotides, and measuring a mean length of each of the plurality of microsatellite repeat elements from a blood sample of a subject based on the analysis of the tumor DNA. For example, MSI of a subject may be assessed by identifying the presence or absence of MSI in the subject. An MSI status may be generated from a selected set of repeat elements based on, for example, the measured mean insertion or deletion (indel) lengths of the microsatellite repeat elements relative to either the reference genome or a patient-specific reference length, the fraction of the set of microsatellite repeat elements containing an insertion or deletion (indel) beyond a certain size, such as a deletion of two repeat units, or the mean number of microsatellite lengths in the sequencing data at each microsatellite locus. The MSI status for a subject may be indicative of a diagnosis, prognosis, or treatment selection for a subject.

In some embodiments, an MSI status may vary (e.g., increase or decrease) over a duration of time (e.g., over two or more different time points). In some embodiments, this duration of time may correspond to, e.g., a course of treatment for the cancer of the subject or a monitoring period after surgical resection or other treatment of a tumor for (e.g., to detect recurrence of the tumor in the subject). In some embodiments, generation of an MSI status may comprise generating a quantitative measure of cfDNA sequencing reads for each of a plurality of genetic loci corresponding to microsatellites. The plurality of genetic loci may comprise microsatellites, such as the entire set of microsatellite repeats in the human reference genome (or a subset thereof), a set of microsatellite repeats optimized to minimize noise in microsatellite stable (MSS) data (or a subset thereof), a set of microsatellite repeats all of the same class (such as all repeats whose repeated unit is of length one, or a subset thereof), a set of microsatellite repeat units that are within a certain range of sizes (e.g., lengths), a set of microsatellite repeats where the sequencing data indicate the lack of a confounding germline insertions or deletions (indels) (or a subset thereof), a set of microsatellite repeats optimized to maximize the performance of the algorithm given a set of training data (or a subset thereof), or a union or intersection of a combination thereof. In some cases, the quantitative measure of cfDNA (e.g., sequencing reads) may comprise a count of sequencing reads that align with each of the plurality of genetic loci. Alternatively, obtaining the quantitative measure of cfDNA may comprise performing binding measurements of the plurality of cfDNA molecules at each of the plurality of microsatellite repeat elements. In some embodiments, generation of an MSI status may comprise generating a comparison (e.g., a difference or a ratio) of quantitative measures for cfDNA (e.g., sequencing reads). By assessing a comparison of counts of sequencing reads across different sets of genetic loci corresponding to microsatellites, methods provided herein may allow generation of MSI statuses, which can be useful for diagnosis, prognosis, or treatment selection for a subject through a non-invasive lab test (e.g., a blood-based test).

In an aspect, the present disclosure provides a computer-implemented method of assessing microsatellite instability of a subject, comprising: obtaining a quantitative measure of a plurality of microsatellite repeat elements from a blood sample of a subject; processing the plurality of quantitative measures to obtain a statistical measure of deviation of the plurality of quantitative measures; and detecting a presence of the microsatellite instability (MSI) of the subject when the statistical measure of deviation of the plurality of quantitative measures satisfies a predetermined criterion, or detecting an absence of the microsatellite instability (MSI) of the subject when the statistical measure of deviation of the plurality of quantitative measures does not satisfy the predetermined criterion.

In some embodiments, the quantitative measure of the plurality of microsatellite repeat elements is selected from the group consisting of a mean length at each of the plurality of microsatellite repeat elements (or a subset thereof), a number, frequency, or fraction of the plurality of microsatellite repeat elements having a length that falls within a predetermined size range (or a subset thereof), and a mean insertion or deletion (indel) length of each of the plurality of microsatellite repeat elements (or a subset thereof). In some embodiments, the subject is diagnosed with cancer. In some embodiments, the subject is asymptomatic for cancer. In some embodiments, the subject has one or more risk factors for cancer (e.g., age, sex, race, ethnicity, family history, history of tobacco or alcohol use, presence of genetic variants, or other clinical health characteristics). In some embodiments, the plurality of quantitative measures is measured from a plurality of cell-free DNA (cfDNA) molecules. In some embodiments, the plurality of quantitative measures is measured from a set of sequencing reads at each of the plurality of microsatellite repeat elements in the plurality of cfDNA molecules. In some embodiments, the method further comprises sequencing the plurality of cfDNA molecules to generate the set of sequencing reads. In some embodiments, the sequencing comprises whole genome sequencing (WGS). In some embodiments, the sequencing is performed at a depth of no more than about 50×, no more than about 48×, no more than about 46×, no more than about 44×, no more than about 42×, no more than about 40×, no more than about 38×, no more than about 36×, no more than about 34×, no more than about 32×, no more than about 30×, no more than about 28×, no more than about 24×, no more than about 22×, no more than about 20×, no more than about 18×, no more than about 16×, no more than about 14×, or no more than about 12×. In some embodiments, the sequencing is performed at a depth of no more than about 10×. In some embodiments, the sequencing is performed at a depth of no more than about 8×. In some embodiments, the sequencing is performed at a depth of no more than about 6×. In some embodiments, the sequencing is performed at a depth of no more than about 5×, no more than about 4×, no more than about 3×, no more than about 2×, or no more than about 1×. In some embodiments, measuring the plurality of quantitative measures comprises performing binding measurements of the plurality of cfDNA molecules at each of the plurality of microsatellite repeat elements (or a subset thereof).

In some embodiments, the method further comprises, based on the detected presence or absence of the microsatellite instability of the subject, identifying a treatment for the subject and/or administering a therapeutically effective amount of a treatment to the subject. In some embodiments, the treatment is selected from the group consisting of a chemotherapy, a radiation therapy, and an immunotherapy. In some embodiments, the treatment comprises an immunotherapy. In some embodiments, the immunotherapy comprises pembrolizumab. In some embodiments, the method further comprises enriching the plurality of cfDNA molecules for at least a subset of the plurality of microsatellite repeat elements. In some embodiments, the enrichment comprises amplifying the plurality of cfDNA molecules. In some embodiments, the amplification comprises selective amplification (e.g., targeted PCR, or targeted enrichment followed by universal or targeted PCR). In some embodiments, the amplification comprises universal amplification (e.g., universal PCR). In some embodiments, the enrichment comprises selectively isolating at least a portion of the plurality of cfDNA molecules (e.g., targeted enrichment). In some embodiments, the at least the portion comprises mononucleotides. In some embodiments, the at least the portion comprises dinucleotides.

In some embodiments, the statistical measure of deviation is a mean z-score. In some embodiments, the statistical measure of deviation is a mean z-score relative to a reference blood sample. In some embodiments, the reference blood sample is obtained from a subject having microsatellite instability (e.g., an MSI-positive subject). In some embodiments, the reference blood sample is obtained from a subject not having microsatellite instability (e.g., an MSI-negative or MSS subject). In some embodiments, the predetermined criterion is the absolute value of the mean z-score being greater than a predetermined number. In some embodiments, the predetermined number is about 1. In some embodiments, the predetermined number is about 2. In some embodiments, the predetermined number is about 3. In some embodiments, the plurality of microsatellite repeat elements comprises mononucleotides or dinucleotides. In some embodiments, the plurality of microsatellite repeat elements comprises mononucleotides and dinucleotides.

In some embodiments, the plurality of microsatellite repeat elements comprises at least about 1 million distinct microsatellite repeat elements. In some embodiments, the plurality of microsatellite repeat elements comprises at least about 5 million distinct microsatellite repeat elements. In some embodiments, the plurality of microsatellite repeat elements comprises at least about 10 million distinct microsatellite repeat elements. In some embodiments, the plurality of microsatellite repeat elements comprises at least about 20 million distinct microsatellite repeat elements.

In some embodiments, the presence of the microsatellite instability of the subject is detected with a sensitivity of at least about 70%. In some embodiments, the presence of the microsatellite instability of the subject is detected with a sensitivity of at least about 80%. In some embodiments, the presence of the microsatellite instability of the subject is detected with a sensitivity of at least about 90%. In some embodiments, the presence of the microsatellite instability of the subject is detected with a sensitivity of at least about 95%. In some embodiments, the presence of the microsatellite instability of the subject is detected with a sensitivity of at least about 96%, at least about 97%, or at least about 98%. In some embodiments, the presence of the microsatellite instability of the subject is detected with a sensitivity of at least about 99%.

In some embodiments, the absence of the microsatellite instability of the subject is detected with a specificity of at least about 70%. In some embodiments, the absence of the microsatellite instability of the subject is detected with a specificity of at least about 80%. In some embodiments, the absence of the microsatellite instability of the subject is detected with a specificity of at least about 90%. In some embodiments, the absence of the microsatellite instability of the subject is detected with a specificity of at least about 95%. In some embodiments, the absence of the microsatellite instability of the subject is detected with a specificity of at least about 96%, at least about 97%, or at least about 98%. In some embodiments, the absence of the microsatellite instability of the subject is detected with a specificity of at least about 99%.

In some embodiments, the presence of the microsatellite instability of the subject is detected with a positive predictive value (PPV) of at least about 70%. In some embodiments, the presence of the microsatellite instability of the subject is detected with a positive predictive value (PPV) of at least about 80%. In some embodiments, the presence of the microsatellite instability of the subject is detected with a positive predictive value (PPV) of at least about 90%. In some embodiments, the presence of the microsatellite instability of the subject is detected with a positive predictive value (PPV) of at least about 95%. In some embodiments, the presence of the microsatellite instability of the subject is detected with a positive predictive value (PPV) of at least about 96%, at least about 97%, or at least about 98%. In some embodiments, the presence of the microsatellite instability of the subject is detected with a positive predictive value (PPV) of at least about 99%.

In some embodiments, the absence of the microsatellite instability of the subject is detected with a negative predictive value (NPV) of at least about 70%. In some embodiments, the absence of the microsatellite instability of the subject is detected with a negative predictive value (NPV) of at least about 80%. In some embodiments, the absence of the microsatellite instability of the subject is detected with a negative predictive value (NPV) of at least about 90%. In some embodiments, the absence of the microsatellite instability of the subject is detected with a negative predictive value (NPV) of at least about 95%. In some embodiments, the absence of the microsatellite instability of the subject is detected with a negative predictive value (NPV) of at least about 96%, at least about 97%, or at least about 98%. In some embodiments, the absence of the microsatellite instability of the subject is detected with a negative predictive value (NPV) of at least about 99%.

In some embodiments, the presence or absence of the microsatellite instability of the subject is detected with an area under the curve (AUC) of at least about 0.70. In some embodiments, the presence or absence of the microsatellite instability of the subject is detected with an area under the curve (AUC) of at least about 0.80. In some embodiments, the presence or absence of the microsatellite instability of the subject is detected with an area under the curve (AUC) of at least about 0.90. In some embodiments, the presence or absence of the microsatellite instability of the subject is detected with an area under the curve (AUC) of at least about 0.95. In some embodiments, the presence or absence of the microsatellite instability of the subject is detected with an area under the curve (AUC) of at least about 0.96, at least about 0.97, or at least about 0.98. In some embodiments, the presence or absence of the microsatellite instability of the subject is detected with an area under the curve (AUC) of at least about 0.99.

In some embodiments, the method further comprises detecting the presence of a microsatellite stability (MSS) of the subject when the statistical measure of deviation of the plurality of quantitative measures does not satisfy the predetermined criterion, or detecting the absence of a microsatellite stability (MSS) of the subject when the statistical measure of deviation of the plurality of quantitative measures satisfies the predetermined criterion.

In some embodiments, the presence of the microsatellite stability of the subject is detected with a sensitivity of at least about 70%. In some embodiments, the presence of the microsatellite stability of the subject is detected with a sensitivity of at least about 80%. In some embodiments, the presence of the microsatellite stability of the subject is detected with a sensitivity of at least about 90%. In some embodiments, the presence of the microsatellite stability of the subject is detected with a sensitivity of at least about 95%. In some embodiments, the presence of the microsatellite stability of the subject is detected with a sensitivity of at least about 96%, at least about 97%, or at least about 98%. In some embodiments, the presence of the microsatellite stability of the subject is detected with a sensitivity of at least about 99%.

In some embodiments, the absence of the microsatellite stability of the subject is detected with a specificity of at least about 70%. In some embodiments, the absence of the microsatellite stability of the subject is detected with a specificity of at least about 80%. In some embodiments, the absence of the microsatellite stability of the subject is detected with a specificity of at least about 90%. In some embodiments, the absence of the microsatellite stability of the subject is detected with a specificity of at least about 95%. In some embodiments, the absence of the microsatellite stability of the subject is detected with a specificity of at least about 96%, at least about 97%, or at least about 98%. In some embodiments, the absence of the microsatellite stability of the subject is detected with a specificity of at least about 99%.

In some embodiments, the presence of the microsatellite stability of the subject is detected with a positive predictive value (PPV) of at least about 70%. In some embodiments, the presence of the microsatellite stability of the subject is detected with a positive predictive value (PPV) of at least about 80%. In some embodiments, the presence of the microsatellite stability of the subject is detected with a positive predictive value (PPV) of at least about 90%. In some embodiments, the presence of the microsatellite stability of the subject is detected with a positive predictive value (PPV) of at least about 95%. In some embodiments, the presence of the microsatellite stability of the subject is detected with a positive predictive value (PPV) of at least about 96%, at least about 97%, or at least about 98%. In some embodiments, the presence of the microsatellite stability of the subject is detected with a positive predictive value (PPV) of at least about 99%.

In some embodiments, the absence of the microsatellite stability of the subject is detected with a negative predictive value (NPV) of at least about 70%. In some embodiments, the absence of the microsatellite stability of the subject is detected with a negative predictive value (NPV) of at least about 80%. In some embodiments, the absence of the microsatellite stability of the subject is detected with a negative predictive value (NPV) of at least about 90%. In some embodiments, the absence of the microsatellite stability of the subject is detected with a negative predictive value (NPV) of at least about 95%. In some embodiments, the absence of the microsatellite stability of the subject is detected with a negative predictive value (NPV) of at least about 96%, at least about 97%, or at least about 98%. In some embodiments, the absence of the microsatellite stability of the subject is detected with a negative predictive value (NPV) of at least about 99%.

In some embodiments, the presence or absence of the microsatellite stability of the subject is detected with an area under the curve (AUC) of at least about 0.70. In some embodiments, the presence or absence of the microsatellite stability of the subject is detected with an area under the curve (AUC) of at least about 0.80. In some embodiments, the presence or absence of the microsatellite stability of the subject is detected with an area under the curve (AUC) of at least about 0.90. In some embodiments, the presence or absence of the microsatellite stability of the subject is detected with an area under the curve (AUC) of at least about 0.95. In some embodiments, the presence or absence of the microsatellite stability of the subject is detected with an area under the curve (AUC) of at least about 0.96, at least about 0.97, or at least about 0.98. In some embodiments, the presence or absence of the microsatellite stability of the subject is detected with an area under the curve (AUC) of at least about 0.99.

In another aspect, the present disclosure provides a system, comprising a controller comprising or capable of accessing, a non-transitory computer-readable medium comprising machine-executable instructions which, upon execution by one or more computer processors, perform a method for assessing microsatellite instability of a subject, the method comprising: obtaining a quantitative measure of a plurality of microsatellite repeat elements from a blood sample of a subject; processing the plurality of quantitative measures to obtain a statistical measure of deviation of the plurality of quantitative measures; and detecting a presence of the microsatellite instability (MSI) of the subject when the statistical measure of deviation of the plurality of quantitative measures satisfies a predetermined criterion, or detecting an absence of the microsatellite instability (MSI) of the subject when the statistical measure of deviation of the plurality of quantitative measures does not satisfy the predetermined criterion.

In some embodiments, the quantitative measure of the plurality of microsatellite repeat elements is selected from the group consisting of a mean length at each of the plurality of microsatellite repeat elements (or a subset thereof), a number, frequency, or fraction of the plurality of microsatellite repeat elements having a length that falls within a predetermined size range (or a subset thereof), and a mean insertion or deletion (indel) length of each of the plurality of microsatellite repeat elements (or a subset thereof). In some embodiments, the subject is diagnosed with cancer. In some embodiments, the subject is asymptomatic for cancer. In some embodiments, the subject has one or more risk factors for cancer (e.g., age, sex, race, ethnicity, family history, history of tobacco or alcohol use, presence of genetic variants, or other clinical health characteristics). In some embodiments, the plurality of quantitative measures is measured from a plurality of cell-free DNA (cfDNA) molecules. In some embodiments, the plurality of quantitative measures is measured from a set of sequencing reads at each of the plurality of microsatellite repeat elements in the plurality of cfDNA molecules. In some embodiments, the method of the system further comprises sequencing the plurality of cfDNA molecules to generate the set of sequencing reads. In some embodiments, the sequencing comprises whole genome sequencing (WGS). In some embodiments, the sequencing is performed at a depth of no more than about 50×, no more than about 48×, no more than about 46×, no more than about 44×, no more than about 42×, no more than about 40×, no more than about 38×, no more than about 36×, no more than about 34×, no more than about 32×, no more than about 30×, no more than about 28×, no more than about 24×, no more than about 22×, no more than about 20×, no more than about 18×, no more than about 16×, no more than about 14×, or no more than about 12×. In some embodiments, the sequencing is performed at a depth of no more than about 10×. In some embodiments, the sequencing is performed at a depth of no more than about 8×. In some embodiments, the sequencing is performed at a depth of no more than about 6×. In some embodiments, the sequencing is performed at a depth of no more than about 5×, no more than about 4×, no more than about 3×, no more than about 2×, or no more than about 1×. In some embodiments, measuring the plurality of quantitative measures comprises performing binding measurements of the plurality of cfDNA molecules at each of the plurality of microsatellite repeat elements (or a subset thereof).

In some embodiments, the method of the system further comprises, based on the detected presence or absence of the microsatellite instability of the subject, identifying a treatment for the subject or a therapeutically effective amount of a treatment to be administered to the subject. In some embodiments, the treatment is selected from the group consisting of a chemotherapy, a radiation therapy, and an immunotherapy. In some embodiments, the treatment comprises an immunotherapy. In some embodiments, the immunotherapy comprises pembrolizumab. In some embodiments, the method of the system further comprises directing the enrichment of the plurality of cfDNA molecules for at least a subset of the plurality of microsatellite repeat elements. In some embodiments, the enrichment comprises amplifying the plurality of cfDNA molecules. In some embodiments, the amplification comprises selective amplification (e.g., targeted PCR, or targeted enrichment followed by universal or targeted PCR). In some embodiments, the amplification comprises universal amplification (e.g., universal PCR). In some embodiments, the enrichment comprises selectively isolating at least a portion of the plurality of cfDNA molecules (e.g., targeted enrichment). In some embodiments, the at least the portion comprises mononucleotides. In some embodiments, the at least the portion comprises dinucleotides.

In some embodiments, the statistical measure of deviation is a mean z-score. In some embodiments, the statistical measure of deviation is a mean z-score relative to a reference blood sample. In some embodiments, the reference blood sample is obtained from a subject having microsatellite instability (e.g., an MSI-positive subject). In some embodiments, the reference blood sample is obtained from a subject not having microsatellite instability (e.g., an MSI-negative or MSS subject). In some embodiments, the predetermined criterion is the absolute value of the mean z-score being greater than a predetermined number. In some embodiments, the predetermined number is about 1. In some embodiments, the predetermined number is about 2. In some embodiments, the predetermined number is about 3. In some embodiments, the plurality of microsatellite repeat elements comprises mononucleotides or dinucleotides. In some embodiments, the plurality of microsatellite repeat elements comprises mononucleotides and dinucleotides.

In some embodiments, the plurality of microsatellite repeat elements comprises at least about 1 million distinct microsatellite repeat elements. In some embodiments, the plurality of microsatellite repeat elements comprises at least about 5 million distinct microsatellite repeat elements. In some embodiments, the plurality of microsatellite repeat elements comprises at least about 10 million distinct microsatellite repeat elements. In some embodiments, the plurality of microsatellite repeat elements comprises at least about 20 million distinct microsatellite repeat elements.

In some embodiments, the presence of the microsatellite instability of the subject is detected with a sensitivity of at least about 70%. In some embodiments, the presence of the microsatellite instability of the subject is detected with a sensitivity of at least about 80%. In some embodiments, the presence of the microsatellite instability of the subject is detected with a sensitivity of at least about 90%. In some embodiments, the presence of the microsatellite instability of the subject is detected with a sensitivity of at least about 95%. In some embodiments, the presence of the microsatellite instability of the subject is detected with a sensitivity of at least about 96%, at least about 97%, or at least about 98%. In some embodiments, the presence of the microsatellite instability of the subject is detected with a sensitivity of at least about 99%.

In some embodiments, the absence of the microsatellite instability of the subject is detected with a specificity of at least about 70%. In some embodiments, the absence of the microsatellite instability of the subject is detected with a specificity of at least about 80%. In some embodiments, the absence of the microsatellite instability of the subject is detected with a specificity of at least about 90%. In some embodiments, the absence of the microsatellite instability of the subject is detected with a specificity of at least about 95%. In some embodiments, the absence of the microsatellite instability of the subject is detected with a specificity of at least about 96%, at least about 97%, or at least about 98%. In some embodiments, the absence of the microsatellite instability of the subject is detected with a specificity of at least about 99%.

In some embodiments, the presence of the microsatellite instability of the subject is detected with a positive predictive value (PPV) of at least about 70%. In some embodiments, the presence of the microsatellite instability of the subject is detected with a positive predictive value (PPV) of at least about 80%. In some embodiments, the presence of the microsatellite instability of the subject is detected with a positive predictive value (PPV) of at least about 90%. In some embodiments, the presence of the microsatellite instability of the subject is detected with a positive predictive value (PPV) of at least about 95%. In some embodiments, the presence of the microsatellite instability of the subject is detected with a positive predictive value (PPV) of at least about 96%, at least about 97%, or at least about 98%. In some embodiments, the presence of the microsatellite instability of the subject is detected with a positive predictive value (PPV) of at least about 99%.

In some embodiments, the absence of the microsatellite instability of the subject is detected with a negative predictive value (NPV) of at least about 70%. In some embodiments, the absence of the microsatellite instability of the subject is detected with a negative predictive value (NPV) of at least about 80%. In some embodiments, the absence of the microsatellite instability of the subject is detected with a negative predictive value (NPV) of at least about 90%. In some embodiments, the absence of the microsatellite instability of the subject is detected with a negative predictive value (NPV) of at least about 95%. In some embodiments, the absence of the microsatellite instability of the subject is detected with a negative predictive value (NPV) of at least about 96%, at least about 97%, or at least about 98%. In some embodiments, the absence of the microsatellite instability of the subject is detected with a negative predictive value (NPV) of at least about 99%.

In some embodiments, the presence or absence of the microsatellite instability of the subject is detected with an area under the curve (AUC) of at least about 0.70. In some embodiments, the presence or absence of the microsatellite instability of the subject is detected with an area under the curve (AUC) of at least about 0.80. In some embodiments, the presence or absence of the microsatellite instability of the subject is detected with an area under the curve (AUC) of at least about 0.90. In some embodiments, the presence or absence of the microsatellite instability of the subject is detected with an area under the curve (AUC) of at least about 0.95. In some embodiments, the presence or absence of the microsatellite instability of the subject is detected with an area under the curve (AUC) of at least about 0.96, at least about 0.97, or at least about 0.98. In some embodiments, the presence or absence of the microsatellite instability of the subject is detected with an area under the curve (AUC) of at least about 0.99.

In some embodiments, the method of the system further comprises detecting a presence of a microsatellite stability (MSS) of the subject when the statistical measure of deviation of the plurality of quantitative measures does not satisfy the predetermined criterion, or detecting an absence of a microsatellite stability (MSS) of the subject when the statistical measure of deviation of the plurality of quantitative measures satisfies the predetermined criterion.

In another aspect, the present disclosure provides a non-transitory computer-readable medium comprising machine-executable code that, upon execution by one or more computer processors, implements a method for assessing microsatellite instability of a subject, the method comprising: obtaining a quantitative measure of a plurality of microsatellite repeat elements from a blood sample of a subject; processing the plurality of quantitative measures to obtain a statistical measure of deviation of the plurality of quantitative measures; and detecting a presence of the microsatellite instability (MSI) of the subject when the statistical measure of deviation of the plurality of quantitative measures satisfies a predetermined criterion, or detecting an absence of the microsatellite instability (MSI) of the subject when the statistical measure of deviation of the plurality of quantitative measures does not satisfy the predetermined criterion.

In some embodiments, the quantitative measure of the plurality of microsatellite repeat elements is selected from the group consisting of a mean length at each of the plurality of microsatellite repeat elements (or a subset thereof), a number, frequency, or fraction of the plurality of microsatellite repeat elements having a length that falls within a predetermined size range (or a subset thereof), and a mean insertion or deletion (indel) length of each of the plurality of microsatellite repeat elements (or a subset thereof). In some embodiments, the subject is diagnosed with cancer. In some embodiments, the subject is asymptomatic for cancer. In some embodiments, the subject has one or more risk factors for cancer (e.g., age, sex, race, ethnicity, family history, history of tobacco or alcohol use, presence of genetic variants, or other clinical health characteristics). In some embodiments, the plurality of quantitative measures is measured from a plurality of cell-free DNA (cfDNA) molecules. In some embodiments, the plurality of quantitative measures is measured from a set of sequencing reads at each of the plurality of microsatellite repeat elements in the plurality of cfDNA molecules. In some embodiments, the method of the non-transitory computer-readable medium further comprises sequencing the plurality of cfDNA molecules to generate the set of sequencing reads. In some embodiments, the sequencing comprises whole genome sequencing (WGS). In some embodiments, the sequencing is performed at a depth of no more than about 50×, no more than about 48×, no more than about 46×, no more than about 44×, no more than about 42×, no more than about 40×, no more than about 38×, no more than about 36×, no more than about 34×, no more than about 32×, no more than about 30×, no more than about 28×, no more than about 24×, no more than about 22×, no more than about 20×, no more than about 18×, no more than about 16×, no more than about 14×, or no more than about 12×. In some embodiments, the sequencing is performed at a depth of no more than about 10×. In some embodiments, the sequencing is performed at a depth of no more than about 8×. In some embodiments, the sequencing is performed at a depth of no more than about 6×. In some embodiments, the sequencing is performed at a depth of no more than about 5×, no more than about 4×, no more than about 3×, no more than about 2×, or no more than about 1×. In some embodiments, measuring the plurality of quantitative measures comprises performing binding measurements of the plurality of cfDNA molecules at each of the plurality of microsatellite repeat elements (or a subset thereof).

In some embodiments, the method of the non-transitory computer-readable medium further comprises, based on the detected presence or absence of the microsatellite instability of the subject, identifying a treatment for the subject or a therapeutically effective amount of a treatment to be administered to the subject. In some embodiments, the treatment is selected from the group consisting of a chemotherapy, a radiation therapy, and an immunotherapy. In some embodiments, the treatment comprises an immunotherapy. In some embodiments, the immunotherapy comprises pembrolizumab. In some embodiments, the method of the non-transitory computer-readable medium further comprises directing the enrichment of the plurality of cfDNA molecules for at least a subset of the plurality of microsatellite repeat elements. In some embodiments, the enrichment comprises amplifying the plurality of cfDNA molecules. In some embodiments, the amplification comprises selective amplification (e.g., targeted PCR, or targeted enrichment followed by universal or targeted PCR). In some embodiments, the amplification comprises universal amplification (e.g., universal PCR). In some embodiments, the enrichment comprises selectively isolating at least a portion of the plurality of cfDNA molecules (e.g., targeted enrichment). In some embodiments, the at least the portion comprises mononucleotides. In some embodiments, the at least the portion comprises dinucleotides.

In some embodiments, the statistical measure of deviation is a mean z-score. In some embodiments, the statistical measure of deviation is a mean z-score relative to a reference blood sample. In some embodiments, the reference blood sample is obtained from a subject having microsatellite instability (e.g., an MSI-positive subject). In some embodiments, the reference blood sample is obtained from a subject not having microsatellite instability (e.g., an MSI-negative or MSS subject). In some embodiments, the predetermined criterion is the absolute value of the mean z-score being greater than a predetermined number. In some embodiments, the predetermined number is about 1. In some embodiments, the predetermined number is about 2. In some embodiments, the predetermined number is about 3. In some embodiments, the plurality of microsatellite repeat elements comprises mononucleotides or dinucleotides. In some embodiments, the plurality of microsatellite repeat elements comprises mononucleotides and dinucleotides.

In some embodiments, the plurality of microsatellite repeat elements comprises at least about 1 million distinct microsatellite repeat elements. In some embodiments, the plurality of microsatellite repeat elements comprises at least about 5 million distinct microsatellite repeat elements. In some embodiments, the plurality of microsatellite repeat elements comprises at least about 10 million distinct microsatellite repeat elements. In some embodiments, the plurality of microsatellite repeat elements comprises at least about 20 million distinct microsatellite repeat elements.

In some embodiments, the presence of the microsatellite instability of the subject is detected with a sensitivity of at least about 70%. In some embodiments, the presence of the microsatellite instability of the subject is detected with a sensitivity of at least about 80%. In some embodiments, the presence of the microsatellite instability of the subject is detected with a sensitivity of at least about 90%. In some embodiments, the presence of the microsatellite instability of the subject is detected with a sensitivity of at least about 95%. In some embodiments, the presence of the microsatellite instability of the subject is detected with a sensitivity of at least about 96%, at least about 97%, or at least about 98%. In some embodiments, the presence of the microsatellite instability of the subject is detected with a sensitivity of at least about 99%.

In some embodiments, the absence of the microsatellite instability of the subject is detected with a specificity of at least about 70%. In some embodiments, the absence of the microsatellite instability of the subject is detected with a specificity of at least about 80%. In some embodiments, the absence of the microsatellite instability of the subject is detected with a specificity of at least about 90%. In some embodiments, the absence of the microsatellite instability of the subject is detected with a specificity of at least about 95%. In some embodiments, the absence of the microsatellite instability of the subject is detected with a specificity of at least about 96%, at least about 97%, or at least about 98%. In some embodiments, the absence of the microsatellite instability of the subject is detected with a specificity of at least about 99%.

In some embodiments, the presence of the microsatellite instability of the subject is detected with a positive predictive value (PPV) of at least about 70%. In some embodiments, the presence of the microsatellite instability of the subject is detected with a positive predictive value (PPV) of at least about 80%. In some embodiments, the presence of the microsatellite instability of the subject is detected with a positive predictive value (PPV) of at least about 90%. In some embodiments, the presence of the microsatellite instability of the subject is detected with a positive predictive value (PPV) of at least about 95%. In some embodiments, the presence of the microsatellite instability of the subject is detected with a positive predictive value (PPV) of at least about 96%, at least about 97%, or at least about 98%. In some embodiments, the presence of the microsatellite instability of the subject is detected with a positive predictive value (PPV) of at least about 99%.

In some embodiments, the absence of the microsatellite instability of the subject is detected with a negative predictive value (NPV) of at least about 70%. In some embodiments, the absence of the microsatellite instability of the subject is detected with a negative predictive value (NPV) of at least about 80%. In some embodiments, the absence of the microsatellite instability of the subject is detected with a negative predictive value (NPV) of at least about 90%. In some embodiments, the absence of the microsatellite instability of the subject is detected with a negative predictive value (NPV) of at least about 95%. In some embodiments, the absence of the microsatellite instability of the subject is detected with a negative predictive value (NPV) of at least about 96%, at least about 97%, or at least about 98%. In some embodiments, the absence of the microsatellite instability of the subject is detected with a negative predictive value (NPV) of at least about 99%.

In some embodiments, the presence or absence of the microsatellite instability of the subject is detected with an area under the curve (AUC) of at least about 0.70. In some embodiments, the presence or absence of the microsatellite instability of the subject is detected with an area under the curve (AUC) of at least about 0.80. In some embodiments, the presence or absence of the microsatellite instability of the subject is detected with an area under the curve (AUC) of at least about 0.90. In some embodiments, the presence or absence of the microsatellite instability of the subject is detected with an area under the curve (AUC) of at least about 0.95. In some embodiments, the presence or absence of the microsatellite instability of the subject is detected with an area under the curve (AUC) of at least about 0.96, at least about 0.97, or at least about 0.98. In some embodiments, the presence or absence of the microsatellite instability of the subject is detected with an area under the curve (AUC) of at least about 0.99.

In some embodiments, the method of the non-transitory computer-readable medium further comprises detecting a presence of a microsatellite stability (MSS) of the subject when the statistical measure of deviation of the plurality of quantitative measures does not satisfy the predetermined criterion, or detecting an absence of a microsatellite stability (MSS) of the subject when the statistical measure of deviation of the plurality of quantitative measures satisfies the predetermined criterion.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example method of assessing microsatellite instability in a subject, in accordance with some embodiments.

FIG. 2 shows plots of cumulative density function (CDF, y-axis) versus microsatellite insertion or deletion (indel) length (x-axis) for each of 4 different cohorts of patients: tumor TCGA-A6-A566-01A-11D-A28G, microsatellite stable (MSS) (top left); tumor TCGA-A6-A566-01A-11D-A28G, microsatellite instability high (MSI-H) (top right); tumor TCGA-D7-55, microsatellite stable (MSS) (bottom left); and tumor TCGA-D7-55, microsatellite instability high (MSI-H) (bottom right).

FIG. 3 shows a box plot indicating mean insertion or deletion (indel) lengths of the set of microsatellites assayed from microsatellite stable (MSS) patients (left, in blue) and microsatellite instability high (MSI-H) patients (right, in red).

FIG. 4 shows a box plot indicating mean insertion or deletion (indel) lengths of the set of microsatellites assayed from microsatellite stable (MSS) patients (left, in blue) and microsatellite instability high (MSI-H) patients (right, in red).

FIG. 5 illustrates a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an”, and “the” include plural references unless the context clearly dictates otherwise. For example, the term “a nucleic acid” includes a plurality of nucleic acids, including mixtures thereof.

The term “nucleic acid,” or “polynucleotide,” as used herein, generally refers to a molecule comprising one or more nucleic acid subunits, or nucleotides. A nucleic acid may include one or more nucleotides selected from adenosine (A), cytosine (C), guanine (G), thymine (T) and uracil (U), or variants thereof. A nucleotide generally includes a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups, individually or in combination.

Ribonucleotides are nucleotides in which the sugar is ribose. Deoxyribonucleotides are nucleotides in which the sugar is deoxyribose. A nucleotide can be a nucleoside monophosphate or a nucleoside polyphosphate. A nucleotide can be in an easily incorporated form, such as a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), uridine triphosphate (dUTP) and deoxythymidine triphosphate (dTTP) dNTPs, that include detectable tags, such as luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T, or U, or complementary to a purine (e.g., A or G, or variant thereof) or a pyrimidine (e.g., C, T, or U, or variant thereof). In some examples, a nucleic acid is deoxyribonucleic acid (DNA), ribonucleic acid (RNA), or derivatives or variants thereof. A nucleic acid may be single-stranded or double stranded. A nucleic acid molecule may be linear, curved, or circular or any combination thereof.

The terms “nucleic acid molecule,” “nucleic acid sequence,” “nucleic acid fragment,” “oligonucleotide,” and “polynucleotide,” as used herein, generally refer to a polynucleotide that may have various lengths, such as either deoxyribonucleotides or ribonucleotides (RNA), or analogs thereof. A nucleic acid molecule can have a length of at least about 5 bases, 10 bases, 20 bases, 30 bases, 40 bases, 50 bases, 60 bases, 70 bases, 80 bases, 90, 100 bases, 110 bases, 120 bases, 130 bases, 140 bases, 150 bases, 160 bases, 170 bases, 180 bases, 190 bases, 200 bases, 300 bases, 400 bases, 500 bases, 1 kilobase (kb), 2 kb, 3, kb, 4 kb, 5 kb, 10 kb, or 50 kb, or it may have any number of bases between any two of the aforementioned values. An oligonucleotide is typically composed of a specific sequence of four nucleotide bases: adenine (A); cytosine (C); guanine (G); and thymine (T) (uracil (U) for thymine (T) when the polynucleotide is RNA). Thus, the terms “nucleic acid molecule,” “nucleic acid sequence,” “nucleic acid fragment,” “oligonucleotide,” and “polynucleotide” are at least in part intended to be the alphabetical representation of a polynucleotide molecule. Alternatively, the terms may be applied to the polynucleotide molecule itself. This alphabetical representation can be input into databases in a computer having a central processing unit and/or used for bioinformatics applications such as functional genomics and homology searching. Oligonucleotides may include one or more nonstandard nucleotide(s), nucleotide analog(s) and/or modified nucleotides.

The term “sample,” as used herein, generally refers to a biological sample. Examples of biological samples include nucleic acid molecules, amino acids, polypeptides, proteins, carbohydrates, fats, or viruses. In an example, a biological sample is a nucleic acid sample including one or more nucleic acid molecules. The nucleic acid molecules may be cell-free or cell-free nucleic acid molecules, such as cell-free DNA (cfDNA) or cell-free RNA (cfRNA). The nucleic acid molecules may be derived from a variety of sources including human, mammal, non-human mammal, ape, monkey, chimpanzee, reptilian, amphibian, or avian, sources. Further, samples may be extracted from variety of animal fluids containing cell-free sequences, including but not limited to blood, serum, plasma, vitreous, sputum, urine, tears, perspiration, saliva, semen, mucosal excretions, mucus, spinal fluid, amniotic fluid, lymph fluid and the like. Cell-free polynucleotides (e.g., cfDNA) may be fetal in origin (via fluid taken from a pregnant subject), or may be derived from tissue of the subject itself.

The term “subject,” as used herein, generally refers to an individual having a biological sample that is undergoing processing or analysis. A subject can be an animal or plant. The subject can be a mammal, such as a human, dog, cat, horse, pig, or rodent. The subject can be a patient, e.g., have or be suspected of having a disease, such as one or more cancers (e.g., brain cancer, breast cancer, cervical cancer, colorectal cancer, endometrial cancer, esophageal cancer, gastric cancer, hepatobiliary tract cancer, leukemia, liver cancer, lung cancer, lymphoma, ovarian cancer, pancreatic cancer, skin cancer, urinary tract cancer), one or more infectious diseases, one or more genetic disorder, or one or more tumors, or any combination thereof. For subjects having or suspected of having one or more tumors, the tumors may be of one or more types.

The term “whole blood,” as used herein, generally refers to a blood sample that has not been separated into sub-components (e.g., by centrifugation). The whole blood of a blood sample may contain cfDNA and/or germline DNA. Whole blood DNA (which may contain cfDNA and/or germline DNA) may be extracted from a blood sample. Whole blood DNA sequencing reads (which may contain cfDNA sequencing reads and/or germline DNA sequencing reads) may be extracted from whole blood DNA.

Microsatellite instability (MSI) may generally refer to a condition of genetic predisposition to mutation which may result from impaired DNA mismatch repair (MMR) in a subject. In such subjects, cells with abnormally functioning MMR may accumulate errors during DNA replication, resulting in mutated microsatellite fragments, or repeated DNA sequences. MSI may play a significant role in many types of cancers, such as colon cancer, gastric cancer, endometrial cancer, ovarian cancer, hepatobiliary tract cancer, urinary tract cancer, brain cancer, and skin cancers. For example, MSI is a good marker for detection of hereditary nonpolyposis colorectal cancer (HNPCC) or Lynch syndrome, an autosomal dominant genetic condition that has a high risk of colon cancer and other types of cancers. In addition, microsatellite status may be indicative of a prognosis of a subject for cancer treatments. For example, MSI studies in colon cancer patients have indicated better prognosis for MSI-high patients (MSI-H) as compared to patients with MSI-low (MSI-L) or microsatellite stable (MSS) tumors.

MSI status may be determined according to a method established by the National Cancer Institute (NCI), which may use five microsatellite markers for indication of MSI presence: two mononucleotides (BAT25 and BAT26) and three dinucleotide repeats (D2S123, D5S346, and D17S250). MSI-H tumors may be identified as those with MSI of greater than about 30% of unstable MSI biomarkers, while MSI-L tumors may be identified as those with MSI of less than about 30% of unstable MSI biomarkers.

MSI-L tumors may be classified as tumors of alternative etiologies. Studies may suggest that MSI-H patients respond best to surgery alone, rather than chemotherapy and surgery. An accurate identification of MSI-H status may prevent potentially ineffective treatments such as chemotherapy from being prescribed and administered to patients.

In addition, cancer treatments may be prescribed and administered to patients based at least in part on an identification of MSI in the patient. For example, the U.S. Food and Drug Administration (FDA) has granted accelerated approval to Keytruda™ (pembrolizumab) for adult and pediatric patients with unresectable or metastatic solid tumors characterized by high microsatellite instability or mismatch repair deficiency, after such patients have progressed on alternative drugs. An accurate identification of MSI status may allow accurate clinical decision making, such as prescribing and administering a targeted therapy such as Keytruda™ (pembrolizumab) to patients.

Methods of determining MSI status in patients may comprise tissue analysis. For example, polymerase chain reaction (PCR) and fragment analysis of paired normal and tumor tissue samples may be performed at each of a set of genetic loci (e.g., a standard set of five NCI-recommended loci) to determine microsatellite instability (MSI). The tissue analysis may yield a reported positive test result as MSI-high (indicating that at least two markers are unstable) or a reported negative test result as MSI-low (indicating that one marker is unstable). Such methods of MSI status determination may require an availability of tumor tissue for analysis. In some cases, the availability of tumor tissue may pose challenges. Tissue can be time-consuming and costly to retrieve, requiring coordination with pathologists. Biopsied tissue can be difficult if not impossible to obtain, can be costly and involve painful procedures, and can yield low to moderate clinical relevance due to potential cancer genome evolution. In some cases, a patient's eligibility for Keytruda™ (pembrolizumab) may not be determined until years after an initial cancer diagnosis. Therefore, a liquid biopsy test for determining MSI status may offer advantages of an earlier, less invasive, and less costly alternative to tumor biopsy.

Assessing Microsatellite Instability in DNA Sequence Data from a Subject

Assessment of microsatellite instability (MSI) status may be relatively straightforward when a significant portion (e.g., greater than about 50%, about 60%, about 70%, about 80%, or about 90%) of a sample taken from a subject comes from or is derived from tumor cells. However, in a cell-free DNA (cfDNA) preparation from a subject's plasma derived from a blood sample, the detection of tumor DNA from the cfDNA and the assessment of microsatellite instability (MSI) status therefrom may be an insensitive and noisy process. Detection of tumor DNA and assessment of microsatellite instability (MSI) status from such insensitive and/or noisy signals may be challenging due to the overwhelming signal from non-tumor DNA (e.g., from germline DNA from germline cells that are not tumor derived). The present disclosure provides methods, systems, and media for assessing microsatellite instability (MSI) status from cell-free DNA (cfDNA) sequence data (e.g., cfDNA sequencing reads) or binding measurements of cfDNA molecules derived from a sample of a subject. Once cfDNA sequence data has been received from analysis of a sample from the subject, one or more bioinformatics processes may be used to assess microsatellite instability (MSI) status of the subject.

In an aspect, the present disclosure provides a computer-implemented method for assessing microsatellite instability of a subject, comprising: obtaining a quantitative measure of a plurality of microsatellite repeat elements from a blood sample of a subject; processing the plurality of quantitative measures to obtain a statistical measure of deviation of the plurality of quantitative measures; and detecting a presence of the microsatellite instability (MSI) of the subject when the statistical measure of deviation of the plurality of quantitative measures satisfies a predetermined criterion, or detecting an absence of the microsatellite instability (MSI) of the subject when the statistical measure of deviation of the plurality of quantitative measures does not satisfy the predetermined criterion.

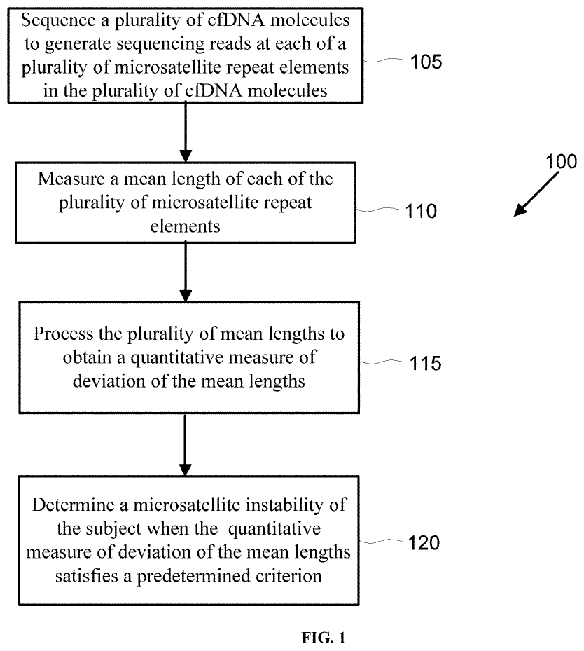

FIG. 1 illustrates an example method of assessing microsatellite instability in a subject, in accordance with some embodiments. In some embodiments, a quantitative measure (e.g., a plurality of mean lengths) is measured from a plurality of cell-free DNA (cfDNA) molecules (as in 105). In some embodiments, measuring the plurality of mean lengths comprises sequencing the plurality of cfDNA molecules to generate sequencing reads at each of the plurality of microsatellite repeat elements in the plurality of cfDNA molecules (as in 110).

For example, sequencing reads may be generated from the cfDNA using any suitable sequencing method. The sequencing method can be a first-generation sequencing method, such as Maxam-Gilbert or Sanger sequencing, or a high-throughput sequencing (e.g., next-generation sequencing or NGS) method. A high-throughput sequencing method may sequence simultaneously (or substantially simultaneously) at least about 10,000, about 100,000, about 1 million, about 10 million, about 100 million, about 1 billion, or more than about 1 billion polynucleotide molecules. Sequencing methods may include, but are not limited to: pyrosequencing, sequencing-by-synthesis, single-molecule sequencing, nanopore sequencing, semiconductor sequencing, sequencing-by-ligation, sequencing-by-hybridization, Digital Gene Expression (Helicos), massively parallel sequencing, e.g., Helicos, Clonal Single Molecule Array (Solexa/Illumina), sequencing using PacBio, SOLiD, Ion Torrent, or Nanopore platforms.

In some embodiments, the sequencing comprises whole genome sequencing (WGS). The sequencing may be performed at a depth sufficient to assess microsatellite instability in a subject with a desired performance (e.g., accuracy, sensitivity, specificity, positive predictive value (PPV), negative predictive value (NPV), or the area under curve (AUC) of a receiver operator characteristic (ROC)). In some embodiments, the sequencing is performed in a “low-pass” manner, for example, at a depth of no more than about 12×, no more than about 11×, no more than about 10×, no more than about 9×, no more than about 8×, no more than about 7×, no more than about 6×, no more than about 5×, no more than about 4×, no more than about 3×, or no more than about 2×.

In some embodiments, assessing microsatellite instability in a subject may comprise aligning the cfDNA sequencing reads to a reference genome. The reference genome may comprise at least a portion of a genome (e.g., the human genome). The reference genome may comprise an entire genome (e.g., the entire human genome). The reference genome may comprise a database comprising a plurality of genomic regions that correspond to coding and/or non-coding genomic regions of a genome. The database may comprise a plurality of genomic regions that correspond to cancer-associated (or tumor-associated) coding and/or non-coding genomic regions of a genome, such as cancer driver mutations (e.g., single nucleotide variants (SNVs), copy number variants (CNVs), insertions or deletions (indels), fusion genes, and microsatellite repeat elements (such as mononucleotides and/or dinucleotides)). For example, the alignment may be performed using a Burrows-Wheeler algorithm or any other suitable alignment algorithm.

In some embodiments, assessing microsatellite instability in a subject may comprise generating a quantitative measure of the cfDNA sequencing reads for each of a plurality of genetic loci. Quantitative measures of the cfDNA sequencing reads may be generated, such as counts of DNA sequencing reads that are aligned with a given genetic locus (e.g., a microsatellite repeat element). CfDNA sequencing reads having a portion or all of the sequencing read aligning with a given microsatellite repeat element may be counted toward the quantitative measure for that microsatellite repeat element.

In some embodiments, the plurality of microsatellite repeat elements is selected from the group consisting of the entire set of microsatellite repeats in the human reference genome (or a subset thereof), a set of microsatellite repeats optimized to minimize noise in MSS data (or a subset thereof), a set of microsatellite repeats all of the same class such as all repeats whose repeated unit is of length one, a set of microsatellite repeat units that are within a certain range of sizes (e.g., lengths), a set of microsatellite repeats where the sequencing data indicate the lack of a confounding germline indel, a set of microsatellite repeats optimized to maximize the performance of the algorithm given a set of training data (or a subset thereof), or a union or intersection of a combination thereof. Patterns of specific and non-specific microsatellite repeat elements may be indicative of microsatellite instability (MSI) status or microsatellite stability (MSS) status. Changes over time in these patterns of microsatellite repeat elements may be indicative of changes in microsatellite instability (MSI) status or microsatellite stability (MSS) status.

In some embodiments, measuring the plurality of mean lengths comprises performing binding measurements of the plurality of cfDNA molecules at each of the plurality of microsatellite repeat elements. In some embodiments, performing the binding measurements comprises assaying the plurality of cfDNA molecules using probes that are selective for at least a portion of the plurality of microsatellite repeat elements in the plurality of cfDNA molecules. In some embodiments, the probes are nucleic acid molecules having sequence complementarity with nucleic acid sequences of the plurality of microsatellite repeat elements. In some embodiments, the nucleic acid molecules are primers or enrichment sequences. In some embodiments, the assaying comprises use of array hybridization or polymerase chain reaction (PCR), or nucleic acid sequencing.

In some embodiments, the method further comprises enriching the plurality of cfDNA molecules for at least a portion of the plurality of microsatellite repeat elements. In some embodiments, the enrichment comprises amplifying the plurality of cfDNA molecules. For example, the plurality of cfDNA molecules may be amplified by selective amplification (e.g., by using a set of primers or probes comprising nucleic acid molecules having sequence complementarity with nucleic acid sequences of the plurality of microsatellite repeat elements). Alternatively or in combination, the plurality of cfDNA molecules may be amplified by universal amplification (e.g., by using universal primers). In some embodiments, the enrichment comprises selectively isolating at least a portion (e.g., mononucleotides and/or dinucleotides) of the plurality of cfDNA molecules.

In some embodiments, the method of assessing microsatellite instability in a subject comprises processing the plurality of mean lengths to obtain a quantitative measure (e.g., a statistical measure) of deviation of the mean lengths (as in 115). In some embodiments, the statistical measure of deviation is a mean z-score relative to one or more reference blood samples. The reference blood samples may be obtained from subjects having a microsatellite instability and/or from subjects not having a microsatellite instability. The reference blood samples may be obtained from subjects having a cancer type or from subjects not having a cancer type (e.g., breast cancer, cervical cancer, colorectal cancer, endometrial cancer, esophageal cancer, gastric cancer, hepatobiliary tract cancer, leukemia, liver cancer, lung cancer, lymphoma, ovarian cancer, pancreatic cancer, skin cancer, urinary tract cancer).

In some embodiments, the method of assessing microsatellite instability in a subject further comprises determining a microsatellite instability (MSI) of the subject when the statistical measure of deviation of the mean lengths satisfies a predetermined criterion (as in 120). The statistical measure of deviation may be a mean z-score, or a mean z-score relative to a reference sample or a reference value. In some embodiments, the predetermined criterion is the absolute value of the mean z-score being greater than a predetermined number. The predetermined number may be about 0.1, about 0.2, about 0.3, about 0.4, about 0.5, about 0.6, about 0.7, about 0.8, about 0.9, about 1, about 1.5, about 2, about 2.5, about 3, about 3.5, about 4, about 4.5, about 5, or more than about 5.

In some embodiments, the plurality of microsatellite repeat elements comprises mononucleotides and/or dinucleotides. The plurality of microsatellite repeat elements may comprise at least about 10 distinct microsatellite repeat elements, at least about 50 distinct microsatellite repeat elements, at least about 100 distinct microsatellite repeat elements, at least about 500 distinct microsatellite repeat elements, at least about 1 thousand distinct microsatellite repeat elements, at least about 5 thousand distinct microsatellite repeat elements, at least about 10 thousand distinct microsatellite repeat elements, at least about 50 thousand distinct microsatellite repeat elements, at least about 100 thousand distinct microsatellite repeat elements, at least about 500 thousand distinct microsatellite repeat elements, at least about 1 million distinct microsatellite repeat elements, at least about 2 million distinct microsatellite repeat elements, at least about 3 million distinct microsatellite repeat elements, at least about 4 million distinct microsatellite repeat elements, at least about 5 million distinct microsatellite repeat elements, at least about 10 million distinct microsatellite repeat elements, at least about 15 million distinct microsatellite repeat elements, at least about 20 million distinct microsatellite repeat elements, at least about 25 million distinct microsatellite repeat elements, at least about 30 million distinct microsatellite repeat elements, or more than 30 million distinct microsatellite repeat elements.

In some embodiments, the presence of the microsatellite instability (MSI) of the subject is detected with a sensitivity of at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the absence of the microsatellite instability (MSI) of the subject is detected with a specificity of at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the presence of the microsatellite instability (MSI) of the subject is detected with a positive predictive value (PPV) of at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the absence of the microsatellite instability (MSI) of the subject is detected with a negative predictive value (NPV) of at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the microsatellite instability (MSI) of the subject is detected with an area under curve (AUC) of a receiver operator characteristic (ROC) of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the method of assessing microsatellite instability in a subject further comprises determining the presence of a microsatellite stability (MSS) of the subject when the statistical measure of deviation of the mean lengths does not satisfy the predetermined criterion, or determining the absence of a microsatellite stability (MSS) of the subject when the statistical measure of deviation of the mean length satisfies the predetermined criterion.

In some embodiments, the presence of the microsatellite stability (MSS) of the subject is detected with a sensitivity of at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the absence of the microsatellite stability (MSS) of the subject is detected with a specificity of at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the presence of the microsatellite stability (MSS) of the subject is detected with a positive predictive value (PPV) of at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the absence of the microsatellite stability (MSS) of the subject is detected with a negative predictive value (NPV) of at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the absence of the microsatellite stability (MSS) of the subject is detected with an area under curve (AUC) of a receiver operator characteristic (ROC) of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the subject has been diagnosed with cancer. For example, the cancer may be one or more types, including: brain cancer, breast cancer, cervical cancer, colorectal cancer, endometrial cancer, esophageal cancer, gastric cancer, hepatobiliary tract cancer, leukemia, liver cancer, lung cancer, lymphoma, ovarian cancer, pancreatic cancer, skin cancer, or urinary tract cancer.

In some embodiments, the method further comprises, based on the determined presence or absence of the microsatellite instability of the subject, administering a therapeutically effective amount of a treatment and/or identifying a treatment to treat the microsatellite instability of the subject. In some embodiments, the treatment comprises a chemotherapy, a radiation therapy, or an immunotherapy. For example, the treatment may comprise an immunotherapy, such as Keytruda™ (pembrolizumab).

A microsatellite instability (MSI) or microsatellite stability (MSS) of a subject may be assessed to determine a diagnosis of a cancer, prognosis of a cancer, or an indication of progression or regression of a tumor in the subject. In addition, one or more clinical outcomes may be assigned based on the microsatellite instability (MSI) or microsatellite stability (MSS) assessment or monitoring (e.g., a difference in microsatellite instability (MSI) or microsatellite stability (MSS) status between two or more time points). Such clinical outcomes may include diagnosing the subject with a cancer comprising tumors of one or more types, diagnosing the subject with the cancer comprising tumors of one or more types and stages, prognosing the subject with the cancer (e.g., indicating a clinical course of treatment (e.g., surgery, chemotherapy, radiotherapy, immunotherapy, or other treatment) for the subject, indicating another clinical course of action (e.g., no treatment, continued monitoring such as on a prescribed time interval basis, stopping a current treatment, switching to another treatment), or indicating an expected survival time for the subject.

In some embodiments, the method of assessing microsatellite instability (MSI) of a subject further comprises determining whether the microsatellite instability (MSI) or microsatellite stability (MSS) is greater than a predetermined threshold. The predetermined threshold may be generated by performing the microsatellite instability (MSI) or microsatellite stability (MSS) assessment on one or more samples from one or more control subjects (e.g., patients known to have a certain tumor type, patients known to have a certain tumor type of a certain stage, or healthy subjects not exhibiting any cancer) and identifying a suitable predetermined threshold based on the microsatellite instability (MSI) or microsatellite stability (MSS) assessments of the control samples.

The predetermined threshold may be adjusted based on a desired sensitivity, specificity, positive predictive value (PPV), negative predictive value (NPV), or accuracy of assessing the microsatellite instability (MSI) or microsatellite stability (MSS) status of a subject. For example, the predetermined threshold may be adjusted to be lower if a high sensitivity of assessing the microsatellite instability (MSI) or microsatellite stability (MSS) status of a subject is desired. Alternatively, the predetermined threshold may be adjusted to be higher if a high specificity assessing the microsatellite instability (MSI) or microsatellite stability (MSS) status of a subject is desired. The predetermined threshold may be adjusted so as to maximize the area under curve (AUC) of a receiver operator characteristic (ROC) of the control samples obtained from the control subjects. The predetermined threshold may be adjusted so as to achieve a desired balance between false positives (FPs) and false negatives (FNs) in assessing microsatellite instability (MSI) or microsatellite stability (MSS) of a cancer comprising a tumor of one or more types.

In some embodiments, the method of assessing microsatellite instability (MSI) or microsatellite stability (MSS) further comprises repeating the assessment at a second later time point. The second time point may be chosen for a suitable comparison of microsatellite instability (MSI) or microsatellite stability (MSS) assessment relative to the first time point. Examples of second time points may correspond to a time after surgical resection, a time during treatment administration or after treatment administration to treat the cancer in the subject to monitor efficiency of the treatment, or a time after cancer is undetectable in the subject after treatment to monitor for residual disease or cancer recurrence in the subject.

In some embodiments, the method of assessing microsatellite instability (MSI) or microsatellite stability (MSS) further comprises determining a difference between the first microsatellite instability (MSI) or microsatellite stability (MSS) status and the second microsatellite instability (MSI) or microsatellite stability (MSS) status, which difference is indicative of a progression or regression of a tumor of the subject. Alternatively or in combination, the method may further comprise generating, by a computer processor, a plot of the first microsatellite instability (MSI) or microsatellite stability (MSS) status and the second microsatellite instability (MSI) or microsatellite stability (MSS) status as a function of the first time point and the second time point, which plot is indicative of the progression or regression of the tumor of the subject. For example, the computer processor may generate a plot of the two or more microsatellite instability (MSI) or microsatellite stability (MSS) statuses on a y-axis against the times corresponding to the time of collection for the data corresponding to the two or more microsatellite instability (MSI) or microsatellite stability (MSS) statuses on an x-axis.

A determined difference or a plot illustrating a difference between the first microsatellite instability (MSI) or microsatellite stability (MSS) status and the second microsatellite instability (MSI) or microsatellite stability (MSS) status may be indicative of a progression or regression of a tumor of the subject. If the second microsatellite instability (MSI) or microsatellite stability (MSS) status is larger than the first microsatellite instability (MSI) or microsatellite stability (MSS) status, that difference may indicate, e.g., tumor progression, inefficacy of a treatment to the tumor in the subject, resistance of the tumor to an ongoing treatment, metastasis of the tumor to other sites in the subject, or residual disease or cancer recurrence in the subject. If the second microsatellite instability (MSI) or microsatellite stability (MSS) status is smaller than the first microsatellite instability (MSI) or microsatellite stability (MSS) status, that difference may indicate, e.g., tumor regression, efficacy of a surgical resection of the tumor in the subject, efficacy of a treatment to the tumor in the subject, or lack of residual disease or cancer recurrence in the subject.

After assessing and/or monitoring microsatellite instability (MSI) or microsatellite stability (MSS) status, one or more clinical outcomes may be assigned based on the microsatellite instability (MSI) or microsatellite stability (MSS) status assessment or monitoring (e.g., a difference in microsatellite instability (MSI) or microsatellite stability (MSS) status between two or more time points). Such clinical outcomes may include diagnosing the subject with a cancer comprising tumors of one or more types, diagnosing the subject with the cancer comprising tumors of one or more types and stages, prognosing the subject with the cancer (e.g., indicating a clinical course of treatment (e.g., surgery, chemotherapy, radiotherapy, immunotherapy, or other treatment) for the subject, indicating another clinical course of action (e.g., no treatment, continued monitoring such as on a prescribed time interval basis, stopping a current treatment, switching to another treatment), or indicating an expected survival time for the subject.

### EXAMPLES

### Example 1: MSI Determination by Whole Genome Sequencing from Patient Tumor-Normal Paired Samples

Whole genome sequencing data was collected from about 500 sets of tumor-normal paired tissue samples obtained from subjects who are cancer patients. A set of 1.3 million genetic loci corresponding to the microsatellites assessed were enriched for short repeat units (e.g., mono-nucleotides and di-nucleotides). Mononucleotide repeats may be abundant and mutated more frequently in MSI-H tumors. For each microsatellite, a mean length was measured for each of the tumor-normal paired tissue samples, and the difference in mean length was calculated. Since MSI-H tumor-normal pairs have more deletions in microsatellites, while microsatellite stable (MSS) tumors do not, the measured mean lengths for each microsatellite of a tumor-normal pair were analyzed to determine MSI status of the subjects.

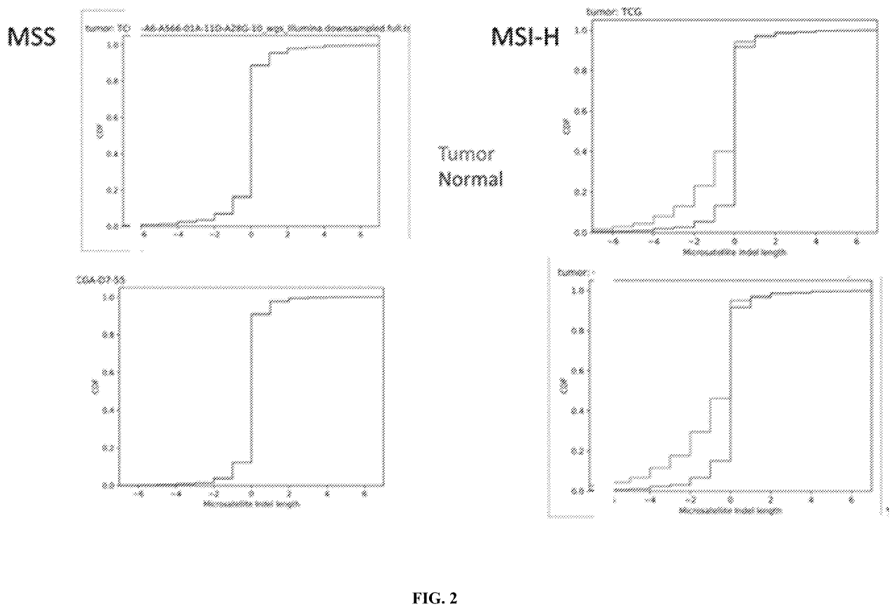

FIG. 2 shows plots of cumulative density function (CDF, y-axis) versus microsatellite insertion or deletion (indel) length (x-axis) for each of 4 different cohorts of patients: tumor TCGA-A6-A566-01A-11D-A28G, microsatellite stable (MSS) (top left); tumor TCGA-A6-A566-01A-11D-A28G, microsatellite instability high (MSI-H) (top right); tumor TCGA-D7-55, microsatellite stable (MSS) (bottom left); and tumor TCGA-D7-55, microsatellite instability high (MSI-H) (bottom right). As shown in FIG. 2, for the two cohorts of patients with MSS status, the measured cumulative density functions (CDFs) indicated that a large majority of the microsatellites measured had an indel length of about zero across both the tumor and normal tissue samples assayed. This result indicated that the MSS tumor-normal pairs had substantially identical microsatellite lengths. In contrast, for the two cohorts of patients with MSI-H status, the measured cumulative density functions (CDFs) indicated that a significant majority of the microsatellites measured had a negative indel length (ranging from about −6 to about 0) of about zero across in the tumor tissue samples assayed. This result indicates that the MSI-H tumor-normal pairs had a statistically significant portion of microsatellites with different microsatellite lengths.

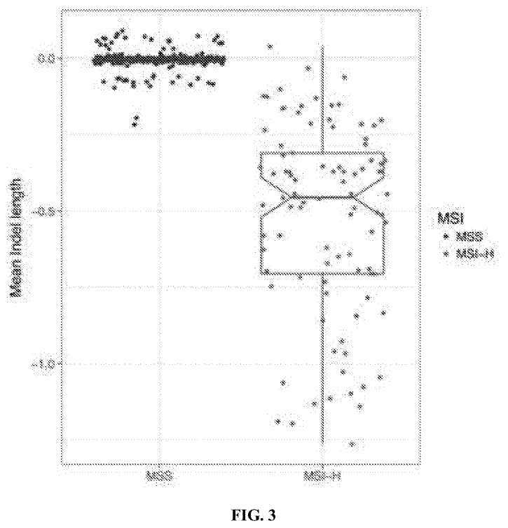

FIG. 3 shows a box plot indicating mean insertion or deletion (indel) lengths of the set of microsatellites assayed from microsatellite stable (MSS) patients (left, in blue) and microsatellite instability high (MSI-H) patients (right, in red). As shown in FIG. 3, for the patients with MSS status, the measured mean indel lengths had a distribution centered around a median of about zero, with a small standard deviation. In contrast, for the patients with MSI-H status, the measured mean indel lengths had a distribution centered around a median of about 0.5, with a significantly larger standard deviation. In particular, nearly all mean indel lengths had absolute values significantly larger than zero. Samples were considered as MSI-H if their mean indel length has a z-score that is less than about −3 (e.g., has an absolute value greater than a predetermined threshold of about 3). The MSI status of the patients were determined based on next-generation sequencing (NGS) data obtained by whole genome sequencing (WGS) of tissue with a high sensitivity of about 98.9% and a high specificity of 93.1%.

### Example 2: MSI Determination by Whole Genome Sequencing from Patient Blood Samples

Whole genome sequencing data is collected from about sets of blood samples obtained from subjects who are cancer patients. Blood samples are collected from patients for analysis of cell-free DNA (cfDNA) to assay circulating tumor DNA (ctDNA) for microsatellite instability status. A set of 1.3 million genetic loci corresponding to the microsatellites assessed are enriched for short repeat units (e.g., mono-nucleotides and di-nucleotides). Mononucleotide repeats may be abundant and mutated more frequently in MSI-H tumors. For each microsatellite, a mean length is measured for each of the blood samples. Since MSI-H tumor-normal pairs have more deletions in microsatellites, while microsatellite stable (MSS) tumors do not, the measured mean lengths for each microsatellite of a blood sample can be analyzed to determine the MSI status of the subjects.

Whole genome sequencing data obtained by performing next-generation sequencing (NGS) of blood samples obtained from patients was simulated by spiking in silico 1% of sequencing reads obtained from tumor tissue into patient-matched normal background reads (e.g., sequencing reads obtained from normal tissue of a tumor-normal paired sample of a subject). The differences in microsatellite lengths were observed even at low tumor fractions (e.g., such as those which tend to be observed in blood), thereby enabling MSI-H and MSS statuses to be distinguished in subjects.

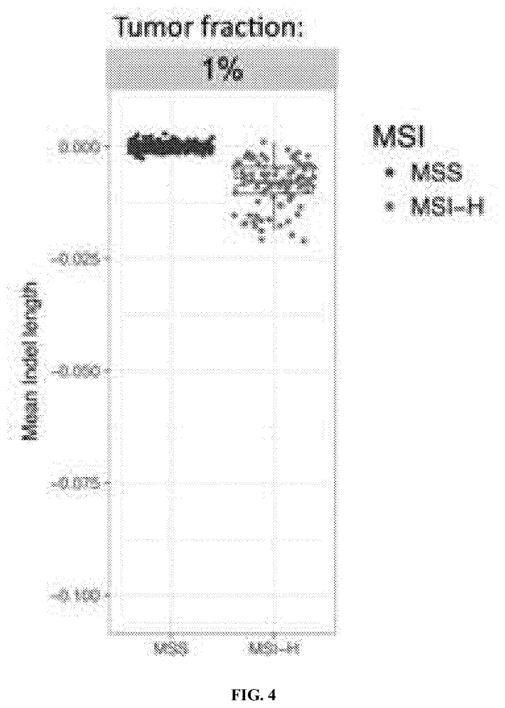

FIG. 4 shows a box plot indicating mean insertion or deletion (indel) lengths of the set of microsatellites assayed from microsatellite stable (MSS) patients (left, in blue) and microsatellite instability high (MSI-H) patients (right, in red). As shown in FIG. 4, for the patients with MSS status, the measured mean indel lengths had a distribution centered around a median of about zero, with a small standard deviation. In contrast, for the patients with MSI-H status, the measured mean indel lengths had a distribution centered around a median of about 0.01, with a significantly larger standard deviation. In particular, nearly all mean indel lengths had absolute values significantly larger than zero. Samples were considered as MSI-H if their mean indel length had a z-score that has an absolute value greater than a predetermined threshold. The MSI status of the patients were determined based on in silico simulated sequencing data measured from blood samples with a low 1% tumor fraction with a high sensitivity of 95.7%, a high specificity of 99.1%, and a classification gap of 1.7.

### Computer Systems

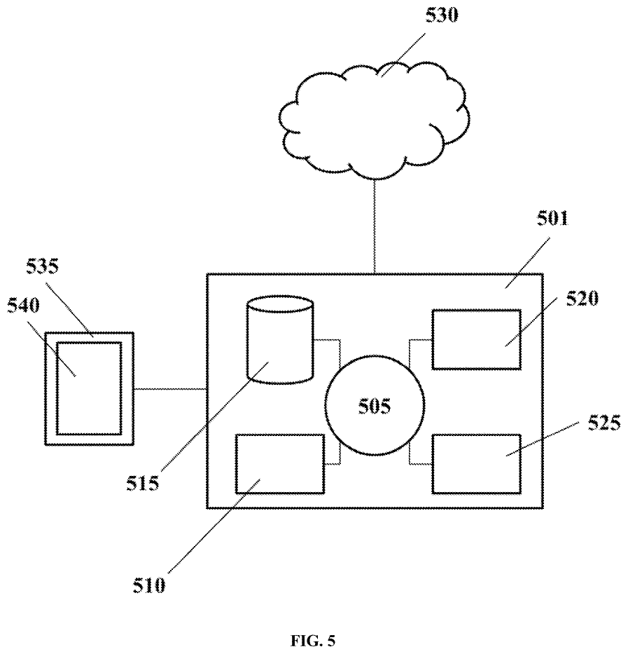

The present disclosure provides computer systems that are programmed to implement methods of the disclosure. FIG. 5 shows a computer system 501 that is programmed or otherwise configured to, for example, obtain a quantitative measure of microsatellite repeat elements from a blood sample of a subject, process the quantitative measures to obtain a statistical measure of deviation of the quantitative measures, and detect a presence of a microsatellite instability (MSI) of the subject when the statistical measure of deviation of the quantitative measures satisfies a predetermined criterion, or detect an absence of the microsatellite instability (MSI) of the subject when the statistical measure of deviation of the plurality of quantitative measures does not satisfy the predetermined criterion. The computer system 501 can regulate various aspects of analysis, calculation, and generation of the present disclosure, such as, for example, obtaining a quantitative measure of microsatellite repeat elements from a blood sample of a subject, processing the quantitative measures to obtain a statistical measure of deviation of the quantitative measures, and detecting a presence of a microsatellite instability (MSI) of the subject when the statistical measure of deviation of the quantitative measures satisfies a predetermined criterion, or detecting an absence of the microsatellite instability (MSI) of the subject when the statistical measure of deviation of the plurality of quantitative measures does not satisfy the predetermined criterion. The computer system 501 can be an electronic device of a user or a computer system that is remotely located with respect to the electronic device. The electronic device can be a mobile electronic device.

The computer system 501 includes a central processing unit (CPU, also “processor” and “computer processor” herein) 505, which can be a single core or multi core processor, or a plurality of processors for parallel processing. The computer system 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 can be a data storage unit (or data repository) for storing data. The computer system 501 can be operatively coupled to a computer network (“network”) 530 with the aid of the communication interface 520. The network 530 can be the Internet, an internet and/or extranet, or an intranet and/or extranet that is in communication with the Internet. The network 530 in some cases is a telecommunication and/or data network. The network 530 can include one or more computer servers, which can enable distributed computing, such as cloud computing. For example, one or more computer servers may enable cloud computing over the network 530 (“the cloud”) to perform various aspects of analysis, calculation, and generation of the present disclosure, such as, for example, obtaining a quantitative measure of microsatellite repeat elements from a blood sample of a subject, processing the quantitative measures to obtain a statistical measure of deviation of the quantitative measures, and determining a microsatellite instability of the subject when the statistical measure of deviation of the quantitative measures satisfies a predetermined criterion. Such cloud computing may be provided by cloud computing platforms such as, for example, Amazon Web Services (AWS), Microsoft Azure, Google Cloud Platform, and IBM cloud. The network 530, in some cases with the aid of the computer system 501, can implement a peer-to-peer network, which may enable devices coupled to the computer system 501 to behave as a client or a server.

The CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and writeback.

The CPU 505 can be part of a circuit, such as an integrated circuit. One or more other components of the system 501 can be included in the circuit. In some cases, the circuit is an application specific integrated circuit (ASIC).

The storage unit 515 can store files, such as drivers, libraries and saved programs. The storage unit 515 can store user data, e.g., user preferences and user programs. The computer system 501 in some cases can include one or more additional data storage units that are external to the computer system 501, such as located on a remote server that is in communication with the computer system 501 through an intranet or the Internet.

The computer system 501 can communicate with one or more remote computer systems through the network 530. For instance, the computer system 501 can communicate with a remote computer system of a user (e.g., a physician, a nurse, a caretaker, a patient, or a subject). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 501 via the network 530.

Methods as described herein can be implemented by way of machine (e.g., computer processor) executable code stored on an electronic storage location of the computer system 501, such as, for example, on the memory 510 or electronic storage unit 515. The machine executable or machine readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 501 can include or be in communication with an electronic display 535 that comprises a user interface (UI) 540 for providing, for example, measured mean lengths of microsatellite repeat elements from a blood sample of a subject, statistical measures of deviation of the mean lengths, and a detected presence or absence of microsatellite instability (MSI) or microsatellite stability (MSS) of the subject. Examples of UIs include, without limitation, a graphical user interface (GUI), and a web-based user interface.

Methods, systems, and media of the present disclosure can be implemented by way of one or more algorithms. An algorithm can be implemented by way of software upon execution by the central processing unit 505. The algorithm can, for example, obtain a quantitative measure of microsatellite repeat elements from a blood sample of a subject, process the quantitative measures to obtain a statistical measure of deviation of the quantitative measures, and detect a presence of a microsatellite instability (MSI) of the subject when the statistical measure of deviation of the quantitative measures satisfies a predetermined criterion, or detect an absence of the microsatellite instability (MSI) of the subject when the statistical measure of deviation of the plurality of quantitative measures does not satisfy the predetermined criterion.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A computer-implemented method for assessing microsatellite instability of a subject, comprising:
obtaining, by one or more processors, a quantitative measure of a plurality of microsatellite repeat elements from a blood sample of a subject;
processing, by the one or more processors, the plurality of quantitative measures to obtain a statistical measure of deviation of the plurality of quantitative measures; and
detecting, by the one or more processors, a presence of the microsatellite instability (MSI) of the subject when the statistical measure of deviation of the plurality of quantitative measures satisfies a predetermined criterion, or detecting an absence of the microsatellite instability (MSI) of the subject when the statistical measure of deviation of the plurality of quantitative measures does not satisfy the predetermined criterion.

2. The method of claim 1, wherein the quantitative measure of the plurality of microsatellite repeat elements is a mean length at each of the plurality of microsatellite repeat elements, a number or fraction of the plurality of microsatellite repeat elements having a length in a predetermined size range, or a mean insertion or deletion (indel) length of each of the plurality of microsatellite repeat elements.

3. The method of claim 1, wherein the subject is diagnosed with cancer.

4. The method of claim 1, wherein the plurality of quantitative measures is measured from a plurality of cell-free DNA (cfDNA) molecules.

5. The method of claim 4, wherein the plurality of quantitative measures is measured from a set of sequencing reads at each of the plurality of microsatellite repeat elements in the plurality of cfDNA molecules.

6. The method of claim 5, further comprising sequencing the plurality of cfDNA molecules to generate the set of sequencing reads.

7. The method of claim 5, wherein the sequencing comprises whole genome sequencing (WGS).

8-10. (canceled)

11. The method of claim 4, wherein measuring the plurality of quantitative measures comprises performing binding measurements of the plurality of cfDNA molecules at each of the plurality of microsatellite repeat elements.

12. The method of claim 1, further comprising, based on the detected presence or absence of the microsatellite instability of the subject, identifying a treatment for the subject or administering a therapeutically effective amount of a treatment to the subject.

13. The method of claim 12, wherein the treatment is a chemotherapy, a radiation therapy, r an immunotherapy.

14. (canceled)

15. (canceled)

16. The method of claim 4, further comprising enriching the plurality of cfDNA molecules for at least a subset of the plurality of microsatellite repeat elements.

17. The method of claim 16, wherein the enrichment comprises; (a) amplifying the plurality of cfDNA molecules, or (b) selectively isolating at least a portion of the plurality of cfDNA molecules.

18-22. (canceled)

23. The method of claim 1, wherein the statistical measure of deviation is a mean z-score.

24. The method of claim 1, wherein the statistical measure of deviation is a mean z-score relative to a reference blood sample.

25. The method of claim 24, wherein the reference blood sample is obtained from a subject having microsatellite instability.

26. The method of claim 24, wherein the reference blood sample is obtained from a subject not having microsatellite instability.

27. The method of claim 23, wherein the predetermined criterion is the absolute value of the mean z-score being greater than a predetermined number.

28. (canceled)

29. The method of claim 1, wherein the plurality of microsatellite repeat elements comprises mononucleotides or dinucleotides.

30. The method of claim 29, wherein the plurality of microsatellite repeat elements comprises mononucleotides and dinucleotides.

31. The method of claim 1, wherein the plurality of microsatellite repeat elements comprises at least about 1 million distinct microsatellite repeat elements.

32-34. (canceled)

35. The method of claim 1, wherein the presence or absence of the microsatellite instability of the subject is detected with a sensitivity of at least about 90%.

36-40. (canceled)

41. The method of claim 1, wherein the presence of the microsatellite instability of the subject is detected with a positive predictive value (PPV) of at least about 90%.

42. The method of claim 1, wherein the presence or absence of the microsatellite instability of the subject is detected with an area under the curve (AUC) of at least about 0.90.

43. The method of claim 1, further comprising detecting a presence of a microsatellite stability (MSS) of the subject when the statistical measure of deviation of the plurality of quantitative measures does not satisfy the predetermined criterion.

44. A system, comprising a controller comprising or capable of accessing, a non-transitory computer-readable medium comprising machine-executable instructions which, upon execution by one or more computer processors, perform a method for assessing microsatellite instability of a subject, the method comprising:
obtaining a quantitative measure of a plurality of microsatellite repeat elements from a blood sample of a subject;
processing the plurality of quantitative measures to obtain a statistical measure of deviation of the plurality of quantitative measures; and
detecting a presence of the microsatellite instability (MSI) of the subject when the statistical measure of deviation of the plurality of quantitative measures satisfies a predetermined criterion, or detecting an absence of the microsatellite instability (MSI) of the subject when the statistical measure of deviation of the plurality of quantitative measures does not satisfy the predetermined criterion.

45-86. (canceled)

87. A non-transitory computer-readable medium comprising machine-executable code that, upon execution by one or more computer processors, implements a method for assessing microsatellite instability of a subject, the method comprising:
obtaining a quantitative measure of a plurality of microsatellite repeat elements from a blood sample of a subject;
processing the plurality of quantitative measures to obtain a statistical measure of deviation of the plurality of quantitative measures; and
detecting a presence of the microsatellite instability (MSI) of the subject when the statistical measure of deviation of the plurality of quantitative measures satisfies a predetermined criterion, or detecting an absence of the microsatellite instability (MSI) of the subject when the statistical measure of deviation of the plurality of quantitative measures does not satisfy the predetermined criterion.

88-129. (canceled)
